(19) United States
(12) Patent Application Publication
(10) Pub. No.: US 2010/0034816 A1
(43) Pub. Date: Feb. 11, 2010

(54) Treatment of Diabetes by at Least One Epidermal Growth Factor Receptor Specific Antibody or a Derivative Thereof

(75) Inventors: Edgar Selzer, St. Andra-Wordern (AT); Gabriela Kornek, Vienna (AT)

Correspondence Address: FULBRIGHT & JAWORSKI L.L.P., 600 CONGRESS AVE., SUITE 2400, AUSTIN, TX, 78701, US

(73) Assignee: NOVELIX THERAPEUTICS GMBH, Vienna (AT)

(21) Appl. No.: 12/520,815

(22) PCT Filed: Dec. 21, 2007
(86) PCT No.: PCT/AT2007/000584

§ 371 Date: Jun. 22, 2009

(30) Foreign Application Priority Data
Dec. 22, 2006 (AT) A 2135/2006

(51) Int. Cl. A61K 39/395; A61P 3/10

(52) U.S. Cl. 424/133.1; 424/130.1

(57) ABSTRACT

The present invention relates to the use of at least one epidermal growth factor receptor specific antibody or a derivative thereof for the manufacture of a medicament for the treatment of diabetes, in particular of the advanced insulin-dependent stage of diabetes mellitus Type 1 and 2 in humans as well as in animals.

The present invention relates to means and methods for the treatment of diabetes, in particular diabetes mellitus.

Diabetes mellitus is characterized in two broad groups based on clinical manifestations, namely, the non-insulin-dependent or maturity onset form, also known as type 2, and the insulin-dependent or juvenile onset form, also known as type 1 diabetes. Clinically, the majority of type 2 maturity onset diabetic patients are obese, with clinical symptoms usually appearing not before an age of 40. In contrast, type 1, juvenile onset patients are usually not over-weight relative to their age and height, but exhibit a rapid onset of the disease at an early age, often before 30. In principle, though, type 1 diabetes can occur at any age. Current therapeutic regimens for type 1 diabetes include modifications to the diet in order to minimize hyperglycemia resulting from the lack of natural insulin, which in turn, is the result of reduced synthesis of insulin by the pancreatic beta cells. Diet is also modified with regard to insulin administration to counter the hypoglycemic effects of the hormone. Whatever the form of treatment, administration of insulin is required for all type 1 diabetics (“insulin-dependent” diabetes).

The pathogenesis of type 2 diabetes involves the development of insulin resistance associated with compensatory hyperinsulinaemia followed by progressive beta-cell impairment that results in decreased insulin secretion and consecutive hyperglycemia. Current therapies ultimately fail to control blood sugar level after 3-5 years. Patients with type 2 diabetes often benefit initially from measures to improve insulin sensitivity such as weight loss, dietary changes, and exercise. Later, the use of oral insulin secretagogues and insulin sensitizers as monotherapy and in combination helps maintain glycemia for varying periods of time. Ultimately, because of the progressive nature of the disease and the progressive decline in pancreatic beta-cell function, insulin therapy is almost always obligatory to achieve optimal glycemic goals. This is due to the progressive damage to the beta cells during the course of the disease and insulin is finally required in most type 2 diabetic patients for the advanced stages of the disease, which is characterized by the development of a dependency on insulin treatment.

Dietary modification is fundamental to the long-term treatment of all forms of diabetes mellitus. In the case of type 1 diabetes mellitus there is a requirement to balance the amount of carbohydrate with the insulin dose at any meal, which in turn is affected by the amount of exercise performed.

Currently, several drug therapies are used for the treatment of diabetes mellitus type 2. Glucosidase inhibitors, such as acarbose, may help to reduce post-prandial peaks of serum glucose, but have major gastrointestinal side effects. The effects of the soluble form of pramlintide on gastric emptying (and, thus, slowing glucose absorption) in type 1 diabetes mellitus have been studied. Agents such as the pancreatic lipase inhibitor orlistat may aid in the reduction of obesity. For the obese, metformin or the recently introduced PPARg agonists thiazolidinediones, e.g. rosiglitazone, may help to improve insulin resistance. Metformin is the drug of first choice for the oral treatment of type 2 diabetes mellitus, once possible contraindications having been excluded. Adjunctive therapies may be needed for additional metabolic problems such as hyperlipidemia or for the treatment of systemic hypertension that is often accompanied by type 2 diabetes mellitus. Adjunct treatments may therefore also be combined with EGFR inhibition.

Specific treatments are being developed to prevent the complications of diabetes mellitus. These include orally active inhibitors of aldose reductase, inhibitors of non-enzymatic glycation such as aminoguanidine or the protein kinase C inhibitor LY333531. Ranirestat is an orally available aldose reductase inhibitor under development.

However, insulin therapy is still the method of choice to treat diabetes mellitus type 2 in the advanced stage, after conventional oral medications fail to be effective. Replacement insulin is generally injected subcutaneously. Absorption of subcutaneously administered insulin is slow, extremely variable and dependent on multiple factors including the site of administration, capillary density, temperature, blood flow and the method used to reduce its absorption rate. The vast majority of modifications of insulin have, to date, involved the use of materials such as zinc or proteins such as protamine to slow absorption.

Recently, molecular modifications of the insulin sequence using site-directed mutagenesis have been utilized to create human insulins (e.g. human insulin lispro) with structures that have a decreased tendency to form oligomers. The absorption of these novel insulins is much more rapid, less variable and as a result improves post-prandial control of glucose.

In the treatment of diabetes mellitus drugs which influence the insulin production and secretion are regularly used. For instance, a wide variety of sulfonylureas is used which act on the sulfonylurea receptor of the K⁺-ATPase channel to increase insulin secretion. They all bind strongly to albumin, vary in cost and duration of action and are best used in those patients where insulin resistance due to obesity has to be addressed. They have serious side-effects such as weight gain and hypoglycaemia. Novel sulfonylureas have greater potency but there is little evidence that they have any greater maximal effect on insulin secretion and improved clinical benefit.

In Costa D B et al. (Diabetes Care 29 (7) (2006):1711), the use of erlotinib as an inhibitor of the EGFR tyrosine kinase activity to partially meliorate the condition of an individual suffering from type 2 non-insulin dependent diabetes mellitus is described, whereby erlotinib had to be administered daily over four weeks to achieve a clinical benefit. This benefit, however, was only the loss of dependency to one of two medicaments used for the treatment of the described patient's disease. Notably, the patient still had to continue taking daily doses of 30 mg of pioglitazone to keep his diabetes under control.

US 2006/058341 relates to thiazolopyridines which are used to inhibit EGFR tyrosine kinase.

U.S. Pat. No. 6,706,721 relates to erlotinib mesylate used to inhibit EGFR tyrosine kinase. According to said US patent, erlotinib mesylate can be used to treat vascular damages occuring in individuals suffering from diabetes mellitus.

In Benter I F et al. (Brit J Pharm 145 (2005):829-836) the use of genistein to treat vascular defects in diabetic animals is described.

It is an object of the present invention to provide new pharmaceutical formulations which may be used to treat efficiently and sustainably diabetes, in particular insulin dependent diabetes mellitus. The new pharmaceutical formulation may be used solely or in addition to conventional diabetes treatments.

Therefore, the present invention relates to the use of at least one epidermal growth factor receptor (EGFR) specific antibody or derivative thereof (e.g. antibody fragment) for the manufacture of a medicament for the treatment or delaying the progress of diabetes, in particular of the advanced insulin-dependent stage of diabetes mellitus type 1 and 2 in humans as well as in animals. Furthermore, the present invention relates to the use of at least one epidermal growth factor receptor specific antibody or a derivative thereof for the manufacture of a medicament for the treatment of non-insulin dependent stages of diabetes mellitus in humans as well as in animals. It surprisingly turned out that the use of epidermal growth factor receptor (EGFR) specific antibody or a derivative thereof allows to effectively treat individuals suffering from diabetes. Most notably, this unexpected treatment concept can be successfully employed through only 1, preferably 2, more preferably 3, even more preferably 5 EGFR antibody administrations in a patient with advanced insulin-dependent diabetes mellitus, for whom no treatment options other than insulin were available previously.

The medicament according to the present invention may, however, also be used to delay the progress of diabetes.

The epidermal growth factor receptor (EGFR also known as ErbB1, HER or EGFR) was the first receptor identified of the ErbB family of receptors. Since then, the ErbB family proteins have increased to four, including EGFR-1 itself (HER-1, ErbB1), HER-2/neu (ErbB2), HER-3 (ErbB3) and HER-4 (ErbB-4). Consequently, in the context of the present invention, the terms “EGFR” and “epidermal growth factor receptor” refer always to all four family members, namely EGFR-1 (HER-1; ErbB1), HER-2 (ErbB2), HER-3 (ErbB3) and HER-4 (ErbB-4). As used herein the term “antibody” refers to single chain, two-chain and multi-chain proteins and glycoproteins belonging to the classes of polyclonal, monoclonal, chimeric, and hetero immunoglobulins (monoclonal antibodies being preferred); it also includes synthetic and genetically engineered variants of these immunoglobulins. It also includes antibodies directed against the EGFR generated by active immunization procedures of individuals using EGFR specific antigenic peptide fragments or other types of molecules capable of eliciting specific immune responses, for example EGFR vaccines (Srikala S Sridhar et al, The Lancet Oncology (2003)). “Antibody derivative” includes Fab, Fab′, F(ab′)₂, and Fv fragments, as well as any portion of an antibody having specificity toward a desired target epitope or epitopes. The antibody according to the present invention may be a humanized antibody which is derived from a non-human antibody, typically murine, that retains or substantially retains the antigen-binding properties of the parent antibody but which is less immunogenic in humans. This may be achieved by various methods including (a) grafting only the non-human CDRs onto human framework and constant regions with or without retention of critical framework residues, or (b) transplanting the entire non-human variable domains, but “cloaking” them with a human-like section by replacement of surface residues. Such methods as are useful in practicing the present invention include those disclosed in Jones et al., Morrison et al., Proc. Natl. Acad. Sci USA, 81 (1984):6851-6855; Morrison and Oi, Adv. Immunol. 44 (1988): 65-92; Verhoeyen et al., Science 239 (1988) :1534-1536; Padlan, Molec. Immun. 28 (1991):489-498; Padlan, Molec. Immun. 31 (3) (1994):169-217. The specificity of an antibody or derivative thereof can be determined by methods known in the art (e.g. ELISA, immunohistochemistry, Western blotting).

It is particularly preferred to use at least one epidermal growth factor receptor (EGFR) specific antibody or derivative thereof as the unique or sole active ingredient capable to treat or delay the progress of diabetes or as the unique or sole active ingredient modulating or inhibiting EGFR or prevent the binding of another ligand to EGFR, thus acting as “EGFR inhibitor”.

As used herein, the term “EGFR inhibitor” refers to any substance or any molecule capable to bind directly to the extracellular domain of the EGFR, thereby inhibiting the activity of said receptor. The activity of the receptor may also be reduced (inhibited) by downregulation of the number of the receptor or also by other mechanisms such as antibody-dependent cellular toxicity (ADCC), as has been shown for example for the antibodies cetuximab and MDX-214. Depending upon the type of ligand and the EGFR dimerisation partner, several different signal transduction pathways can be engaged. These pathways include the Ras/Raf/MEK/ERK and PI3K/PDK1/Akt pathways, further the PLC-γ and JAK/STAT pathways.

It has been shown that antitumor activity of cetuximab and matuzumab, two EGFR type I specific antibodies, is mediated by inhibition of Akt and ERK signaling and depends less on inhibition of EGFR phosporylation itself (Yoshida et al., Int J Cancer. 2007 Nov. 21). Differences in the mode of action between EGFR inhibition by using antibodies and EGFR inhibition using tyrosine kinase inhibitors such as erlotinib or gefitinib are the basis for dual-agent targeting of the EGFR (Huang et al, Cancer Res 64 (2004): 5355-62; Mukohara T et al, Journal of the National Cancer Institute 97 (16) (2005)). Antibodies in particular monoclonal antibodies and tyrosine kinase inhibitors clearly differ in their mode of action at target receptor level (Fischel J L et al, British Journal of Cancer 92 (2005):1063-1068). The primary action mechanism for example of C225, a chimeric monoclonal antibody, is a competitive antagonism for EGFR. Independent of the phosphorylation status of the receptor, the EGFR-C225 complex is subsequently internalized. The outcome of the EGFR-C225 complex following internalization is not clearly documented, particularly regarding the stage between degradation and cell membrane recycling of the intact receptor. Tyrosine kinase inhibitors act on the intracellular cytosolic ATP-binding domain of EGFR by inhibiting EGFR autophosphorylation. Depending on the nature of the tyrosine kinase inhibitor, EGFR inhibition can be either reversible, as with ZD839 or OSI-774, or irreversible, as for instance with PD183805. The irreversibility of the inhibition is due to covalent fixation of the drug at the ATP-binding site. In contrast to the approach using antibodies, tyrosine kinase inhibitors are not strictly specific for the ATP pocket of the EGFR; this can be explained by the fact that tyrosine kinase inhibitors are all ATP competitors at the ATP binding site of the tyrosine kinases. Thus, for tyrosine kinase inhibitors, some variable cross reactivity may exist between EGFR and other HER-B family members such as HER-2 (Fischel J L et al, British Journal of Cancer 92 (2005):1063-1068). Clinical responses to tyrosine kinase inhibitors after failure with cetuximab have been reported. (Raez L E, Lopes G, Lilenbaum R “Clinical responses to gefinitib after failure of treatment with cetuximab in advanced non-small-cell lung cancer”, J Clin Oncol 23 (2005): 4244-5). Taken together, mechanistic differences between the two approaches to EGFR inhibition therefore clearly exist (see references above and Rosell R et al, Clin Cancer Res (2006): 7222-31) and is the basis for optimizing the therapeutic synergy between tyrosine kinase inhibitors and antibodies, as has been shown by Hui K. Gan et al. (J Biol Chem 282 (5) (2007):2840-50).

The inhibitor is preferably able to inhibit the EGFR activity for at least 10%, preferably at least 30%, more preferably at least 50%, even more preferably at least 70%, in particular at least 90%.

The activity as well as the expression levels of the EGFR can be determined by various methods, for example by immunohistochemistry, Western blotting or by assessing the phosphorylation status of the EGFR as well as of various protein kinases that are coupled to the EGFR, for example the MapKinase, STAT or the PI-3 Kinase (Sordella R et al. Science 305 (2004): 1163-7; Sebastian S et al. Biochimica et Biophysica Acta—Reviews on Cancer 1766 (2006):120-139; Yoshida et al., Int J Cancer. 2007 Nov. 21).

The suitability of EGFR specific antibodies for the use of the present invention may be examined by using, for instance, suitable diabetes models. These models may include diet-induced obese (DIO) mouse model, zucker diabetic fatty rats (ZDF), goto-Kakazaki rats (GK) and diabetic (db/db) mice (e.g. Zhang B et al. Science 284 (1999):974-7; Unger R H et al. FASEB J. 15 (2001):312-21; Thupari J N et al. Am J Physiol Endocrinol Metab. 287 (2004):E97-E104) as well as diabetes monkey models (Srinivasan K. et al. Indian J Med Res 125, March 2007, pp 451-472).

EGFR specific antibodies may be administered to an individual suffering or at risk for suffering diabetes, in particular diabetes mellitus, in an amount of 1 to 1000 mg, preferably up to 3000 or 5000 mg, per day. The medicament of the present invention, may be administered up to three or four times a day or up to once a week. The administration period may last from 1 day to 1 month and even years, depending on the progress of the disease. It is particularly preferred to administer the medicament of the present invention (the medicament comprising preferably EGFR specific antibodies or derivatives thereof and/or EGFR inhibitors) from 1 to 14 days daily to 4 times a day in intervals of 1 to 6 months. This means that a certain dose may be administered for a certain period of time after which the medication is interrupted and continued when required or after a defined period of time.

The epidermal growth factor receptor specific antibody is preferably a EGFR type I, EGFR type II, EGFR type III and/or EGFR type IV specific antibody and more preferably selected from the group of antibodies consisting of cetuximab (Merck), matuzumab (Merck), panitumumab (Abgenix/Amgen), pertuzumab (2C4) (Genentech/Roche), trastazumab (Genentech), MDX-447, MDX-H210, MDX214 (Medarex), TheraCIM hR-3 (YM BioSciences/CIMYM Inc), ABX-EGF, EMD72000, Y10, MAb528 plus Rnase and cetuximab/ricin A and combinations thereof. Antibodies directed against the mutated EGFRvIII, for example Mab806 (Life Science Pharmaceuticals), ICR62, Y10 and Ua30:2 may also be used. ErbB receptor inhibitors according to the present invention include, also monoclonal antibodies such as AR-209 (Aronex Pharmaceuticals Inc. of The Woodlands, USA) and 2B-1 (Chiron), and ErbB inhibitors such as those described in U.S. Pat. No. 7,141,576; U.S. Pat. No. 5,587,458; U.S. Pat. No. 5,877,305 and U.S. Pat. No. 6,465,449.

Table A: Examples for major anti-EGFR type I and type II antibodies in preclinical or clinical use (in this table, EGFR means EGFR type I and EGFR type II corresponds to HER-2) are taken from Sebastian S et al. Biochimica et Biophysica Acta—Reviews on Cancer 1766 (2006):120-139 and from Srikala S Sridhar et al, The Lancet Oncology (2003)

| Monoclonal antibody | Properties |
|---|---|
| cetuximab | Anti-EGFR |
| ABX-EGF | Anti-EGFR |
| EMD72000 | Anti-EGFR |
| MAb ICR62 | Anti-EGFR |
| h-R3 | Anti-EGFR |
| MDX-447 | Bispecific, Anti-EGFR |
| MDX-H210 | Bispecific, Anti-HER2 |
| MDX-214 | Anti-EGFR |
| trastuzumab | Anti-HER2 |
| 2C4 | Anti-HER2 |
| Y10 | Anti-EGFRvIII |
| Ua30:2 | Anti-EGFRvIII |
| MAb806 | Anti-EGFRvIII |
| MAb528 plus Rnase | Anti-EGFR |
| cetuximab/ricin A | Anti-EGFR |

EGFR antibodies can be selected from chimerized, humanized, fully human, and single chain antibodies derived from the murine antibody 225 described in U.S. Pat. No. 4,943,533. The most preferably used EGFR antibody is cetuximab which is marketed as Erbitux. The EGFR antibody can also be selected from the antibodies described in U.S. Pat. No. 6,235,883, U.S. Pat. No. 5,558,864,. U.S. Pat. No. 5,891,996, U.S. Pat. No. 7,132,511, U.S. Pat. No. 5,844,093, and U.S. Pat. No. 5,969,107.

The EGFR specific antibody which is able to bind to the extracellular domain of the EGFR receptor may be of any type, provided that said antibody is able to compete with naturally occurring ligands (e.g. epidermal growth factor, transformation growth factor α (TGFα), neuregulin (neu), and others) which stimulate the receptor. Therefore, the EGFR antibody preferably has a higher affinity to the receptor than other receptor ligands stimulating said receptor (e.g. in particular naturally occurring and EGFR binding ligands). Competition of an antibody with the ligand and thus inhibiting the activation of the receptor may also occur by directly binding the ligand before it binds to and activates the cognate receptor. It turned out that antibodies are particularly suited to be used to bind to EGFR and to block the receptor. In an especially preferred embodiment of the present invention the EGFR inhibitor is cetuximab.

EGFR exists on the cell surface as inactive monomers and is activated by binding of its specific ligands. On activation, EGFR can pair with another EGFR to form an active homodimer or an EGF-receptor may pair with another member of the ErbB receptor family, such as HER-2/neu, to create a heterodimer. This interaction between different types of EGF-receptors allows for cross-regulation of receptor activities in such a way that binding of a ligand to one receptor type may activate another type of receptor. The binding of the ligand, for example of EGF, stimulates the intrinsic protein-tyrosine kinase activity of EGFR which initiates a signal transduction cascade.

EGFR specific active immunization procedures may be used in diabetes patients instead of applying a passive antibody treatment procedure. The concept of active immunotherapy targeting the EGFR has been described by Hu B et al. (J Immunother (1997). 2005 May-June; 28(3):236-44). The amounts of the EGFR specific antigen or derivative thereof to be administered depend on the kind of administration and are well known to the person skilled in the art. A recent example in the literature for generating cetuximab mimotope-induced anti-EGFR antibodies is provided by Riemer A B et al. (J Natl Cancer Inst. 2005 Nov. 16; 97(22): 1663-70). Vaccination against a mutated form of the EGFR (EGFRvIII) using a EGFRvIII-specific peptide immunization strategy has been demonstrated by Heinerger et al (Clin Cancer Res. 2003 Sep. 15; 9(11):4247-54).

Insulin and insulin derivatives and analogous thereof are regularly used in the treatment of diabetes mellitus. Since the administration of insulin does serve to the body of a patient as a substitution of a deregulated or missing hormone production the efficiency of this treatment is questionable. However, insulin used in combination with EGFR specific antibodies or derivatives thereof according to the present invention, has several advantages. For instance, at the beginning of a diabetes treatment the carbohydrate metabolism is preferably controlled by the addition of extrinsic insulin. In the course of the treatment the amount of insulin present in the medicament may be reduced. In contrast to daily insulin applications, one treatment per week with cetuximab over three weeks, for example, was sufficient to eliminate the use of insulin and to control diabetes in a patient for at least 20 weeks. The insulin and insulin derivative preferably comprised in the medicament of the present invention is preferably selected from the group consisting of insulin (human insulin recombinantely produced; e.g. Humulin), insulin lispro (Humalog; rapid acting), insulin aspart (Novolog; rapid acting), insulin glulisine (Apidra; rapid acting), insulin glargine (Lantus; long acting), insulin detemir (Levemir; intermediate acting), NPH-insulin (Humulin N; intermediate acting), NPL-insulin and combinations thereof. Preferred combinations are among others (see e.g. Mooradian A S Ann Intern Med 145 (2006):125-134):

70% NPH-insulin, 30% regular human insulin

50% NPH-insulin, 50% regular human insulin

75% NPL-insulin, 25% insulin lispro

50% NPL-insulin, 50% insulin lispro

70% insulin protamine aspart, 30% insulin aspart

According to another preferred embodiment of the present invention the medicament is formulated for oral, intravenous, intramuscular, subcutaneous or inhalational administration.

Methods and additives to be used when formulating the medicament of the present invention are known to the person skilled in the art (e.g. “Handbook of Pharmaceutical Manufacturing Formulations” Niazi S K, CRC Press (2004), ISBN: 0849317525). Therefore the medicament may preferably comprise further at least one pharmaceutically acceptable excipient, diluent and/or carrier.

When insulin is present in the medicament, said pharmaceutical formulation is adapted to be administered preferably intravenously, intramuscularly, subcutaneously or inhalationally. Unlike many medicines, insulin cannot be taken orally, because like other proteins it would be broken down in the gastrointestinal tract to its amino acid components.

According to a preferred embodiment of the present invention the medicament comprises 1 to 2000 mg, preferably 1 to 1000 mg, more preferably 10 to 1000 mg, even more preferably 100 to 1000 mg, EGFR specific antibody or derivative thereof.

In order to adapt the pharmaceutical preparation according to the present invention to the dosage forms as outlined above the preparation may comprise preferably further at least one pharmaceutically acceptable excipient, diluent and/or carrier.

The present invention is further illustrated by the following example, however, without being restricted thereto.

### EXAMPLE

### Administration of Cetuximab

A 65-year old male patient with a 21 year history of insulin dependent type 2 diabetes mellitus lost insulin-dependency after combined treatment with Cetuximab (Erbitux) and radiotherapy for locally advanced oropharyngeal cancer. The patient suffered from diabetes-associated long-term complications including peripheral neuropathy and peripheral vascular disease. At the time of cancer diagnosis the patient had a body weight of 64 kg (height 176 cm), a fasting blood glucose level of 224 mg/dl and HbAlc of 7.4%. Cetuximab was administered weekly during radiotherapy (loading dose 400 mg/m² followed by 250 mg/m² weekly). The patient received 100 mg prednisone and antihistaminics before each cetuximab-administration. The patient experienced grade 3 acne-like skin rash, which is a typical side effect of cetuximab; radiation therapy was associated with weight loss of 10 kg. Despite high caloric enteral nutritional support, patient's blood glucose level declined continuously and insulin was discontinued. An oral glucose-tolerance test performed 7 weeks after discontinuation of insulin therapy revealed the following plasma glucose levels: 139 mg (fasting), 192 mg (1 hour), and 235 mg (2 hours); the HbAlc value dropped to 6.1%. Twenty weeks after cetuximab treatment, the fasting blood glucose level was 120 mg and HbAlc 6.1%. The patient did not receive any diabetic medication or diet and his body weight was stable at 57 kg.

1.-9. (canceled)

10. A method of treating diabetes comprising:
obtaining a medicament comprising at least one epidermal growth factor receptor specific antibody or derivative thereof; and
administering the medicament to a human or non-human animal;
wherein diabetes is treated in the human or non-human animal.

11. The method of claim 10, further defined as a method of treating advanced insulin-dependent diabetes mellitus type 1 and/or 2.

12. The method of claim 10, further defined as a method of treating non-insulin dependent stages of diabetes mellitus.

13. The method of claim 10, wherein the at least one epidermal growth factor specific antibody is an EGFR-type I, EGFR-type II, EGFR-type III, and/or EGFR-type IV specific antibody.

14. The method of claim 10, wherein the at least one epidermal growth factor receptor specific antibody is cetuximab, ABX-EGF, EMD72000, MAb ICR62, h-R3, MDX-447, MDX-H210, MDX-214, trastuzumab, 2C4, Y1O, Ua30:2, or Mab806.

15. The method of claim 14, wherein the medicament is further defined as comprising MAb528 plus Rnase and/or cetuximab plus ricin A.

16. The method of claim 10, wherein the medicament further comprises at least one of insulin and/or an insulin derivative.

17. The method of claim 16, wherein the insulin and/or insulin derivative is further defined as insulin lispro, insulin aspart, insulin glulisine, insulin glargine, insulin detemir, NPH-insulin, or NPL-insulin.

18. The method of claim 10, wherein the medicament is formulated for oral, intravenous, intramuscular, subcutaneous, or inhalational administration.

19. The method of claim 10, wherein the medicament further comprises at least one pharmaceutically acceptable excipient, diluent, and/or carrier.

20. The method of claim 10, wherein the medicament comprises 1 to 2000 mg of the epidermal growth factor receptor specific antibody or derivative thereof.

21. The method of claim 20, wherein the medicament comprises 10 to 1000 mg of the epidermal growth factor receptor specific antibody or derivative thereof.

22. The method of claim 21, wherein the medicament comprises 100 to 1000 mg of the epidermal growth factor receptor specific antibody or derivative thereof.

23. The method of claim 10, wherein the medicament is administered daily up to 4 times per day for a period of from 1 to 14 days at intervals of 1 to 6 months.

24. The method of claim 10, wherein the medicament is administered up to 3 or 4 times per day.

25. The method of claim 10, wherein the medicament is administered once a week.

26. The method of claim 10, wherein the medicament is administered for a period lasting from 1 day to 1 month.
